SEQUENCE LISTING (6 sequences)

SEQ ID NO: 1 - DNA, 32 bp, Artificial Sequence
  A 621 bp hla fragment was generated by PCR amplification using primers hlaFor64
ggggggcccgggtatgtcttttccttgtttca

SEQ ID NO: 2 - DNA, 30 bp, Artificial Sequence
  A 621 bp hla fragment was generated by PCR amplification using primers hlaRev684
ggggggcccgggatcaggtagttgcaactg

SEQ ID NO: 3 - DNA, 18 bp, Artificial Sequence
  The 621-bp hla fragment was generated using hla-specific primers.  The antisense hla and sense hla in plasmids pYJ318-7 and pYJ318-16, respectively, were confirmed by the plasmid-specific primer tetRFor1399
caatacattgtaggctgc

SEQ ID NO: 4 - DNA, 19 bp, Artificial Sequence
  After RnaseH treatment, cDNA was used as the template for PCR using the tetR-cat-specific primers, tetRFor1399 and catRev768 and the hla gene-specific primers, hlaFor64 and hlaRev684
ggcaggttagtgacattag

SEQ ID NO: 5 - DNA, 50 bp, Artificial Sequence
  Single-stranded DNA oligonucleotides specific for either sense hla RNA
ggccaggctaaaccacttttgttagcaccttcttcgctataaactctata

SEQ ID NO: 6 - DNA, 49 bp, Artificial Sequence
  Single-stranded DNA oligonucleotides specific for antisense hla RNA
tatagagtttatagcgaagaaggtgctaacaaaagtggtttagcctggc

(19) United States
(12) Patent Application Publication
(10) Pub. No.: US 2005/0095643 A1
(43) Pub. Date: May 5, 2005

(54) Method of generating conditionally expressed mutant cells using expressible antisense sequences

(76) Inventors: Andrea Marra, Menlo Park, CA (US); Martin Rosenberg, Royersford, PA (US); Yinduo Ji, Spring City, PA (US)

Correspondence Address: GLAXOSMITHKLINE;Corporate Intellectual Property - UW2220, P.O. Box 1539, King of Prussia, PA, 19406-0939, US

(21) Appl. No.: 11/017,379

(22) Filed: Dec. 20, 2004

Related U.S. Application Data
(63) Continuation of application No. 10/099,198, filed on Mar. 13, 2002, now abandoned.
Application No. 10/099,198 is a continuation of application No. 09/581,479, filed on May 25, 2000, now abandoned.
(60) Provisional application No. 60/067,446, filed on Dec. 4, 1997.
(60) Provisional application No. 60/082,534, filed on Apr. 20, 1998.
(60) Provisional application No. 60/105,161, filed on Oct. 21, 1998.

(51) Int. Cl. C12Q 1/68; C12N 15/85

(52) U.S. Cl. 435/6; 435/455

(57) ABSTRACT

The present invention provides a method for determining pathogen sensitivity to varying levels of reduction of a gene product using an expression vector system having a promoter that is essentially off in vitro and turns on selectively during the infection process in vivo. Genes and gene products identified by this method as essential to growth of infection of a selected pathogen are also provided. In addition, therapeutic compositions designed to target genes identified by this method are provided.

### RELATED APPLICATIONS

This application claims benefit of U.S. Provisional Application No. 60/067,446 filed Dec. 4, 1997, U.S. Provisional Application No. 60/082,534, filed Apr. 20, 1998, and U.S. Provisional Application No. 60/105,161, filed Oct. 21, 1998.

### FIELD OF THE INVENTION

The present invention provides a method for preparing conditionally expressed gene mutants, including conditional lethal mutants, and a method using such mutants to assess gene essentiality, and compositions useful in such methods. Using this method, gene targets most sensitive to inhibition can be selected for the development of new therapies against selected pathogens.

### BACKGROUND OF THE INVENTION

Identification, sequencing and characterization of genes is a major goal of modern scientific research. By identifying genes, determining their sequences and characterizing their biological function, it is possible to employ recombinant technology to produce large quantities of valuable gene products, e.g. proteins and peptides. Additionally, knowledge of gene sequences can provide a key to diagnosis, prognosis and treatment of a variety of disease states in plants and animals which are due to microbial pathogens and pathogenesis.

A variety of techniques have been described for identifying particular gene sequences on the basis of their gene products. For example, see International Patent Application No. WO91/07087, published May 30, 1991. In addition, methods have been described for the amplification of desired sequences. For example, see International Patent Application No. WO91/17271, published Nov. 14, 1991.

Genes which are essential for the growth of an organism, however, have been difficult to identify in such a manner as to be easily recovered for future analysis. The most common methodology currently employed to identify essential genes is a multi-step process involving the generation of a conditionally lethal mutant pool followed by the screening of duplicate members of that pool under the appropriate permissive and non-permissive conditions. Candidate mutants are then transformed with a genomic library and the desired genes are isolated by complementation of the mutant phenotype. The complementing plasmid is recovered, subcloned, and then retested. However, this procedure comprises multiple subcloning steps to identify and recover the desired genes thus making it both labor intensive and time consuming.

A number of approaches for the isolation of pathogen virulence genes based upon transposon mutagenesis have been developed. These include screening for the loss of specific virulence-associated factors (Lee, et al. J. Infect. Dis. 1987, 156:741), survival within macrophages (Fields, et al Proc. Nat'l Acad. Sci. 1986, 83:5189), and penetration of epithelial cells (Finlay, et al. Mol. Microbiol. 1988, 2:757). However, these methods are restricted to certain stages of infection, and to in vitro systems.

Transposon mutants have also been tested in live animal models of infection (Miller, et al., Infect. Immun., 1989, 57:2758; and Bolker, et al., Mol. Gen. Genet., 1994, 248:547-552). However, comprehensive screening of bacterial genes is not possible due to the inability to identify mutants with attenuated virulence within pools of mutagenized bacteria and thus the huge number of mutants would require individual screening and large numbers of animals.

Hensel, et al have developed an insertional mutagenesis system that uses transposons carrying unique DNA sequence tags for the isolation of bacterial virulence genes (Science, 1995, 269:400-403). In this system, termed signature-tagged mutagenesis (herein “STM”), each transposon mutant is tagged with a different DNA sequence. However, mutants in in vitro essential genes are lost in this system. This permits identification of bacteria recovered from hosts infected with a mixed population of mutants, as well as the negative selection of mutants with attenuated virulence. This method was used to identify virulence genes of Salmonella typhimurium in a murine model of typhoid fever. Further, Slauch, et al. describe a method referred to as IVET which provides a means for identifying transcripts which are essentially absent in vitro, but are on throughout, or during, various phases of infection (Methods in Enzymology 1994, 235:481-492). However, these methods only provide information on the effect of the total absence or the specific up-regulation in vivo of the gene product in the organism and no information on gene essentiality.

Conditional lethal mutants may also be created to abolish gene expression and identify essential genes (de Lorenzo, V, et al., Gene 123:17-24 (1993); Neuwald, A. F., et al., Gene 125: 69-73(1993); and Takiff, H. E., et al., J. Bacteriol. 174:1544-1553(1992). Chemical mutagenesis may also be used to make such mutants (Beckwith, J. Methods in Enzymology 204: 3-18(1991)). These methods of preparing mutants are often time consuming and difficult to reproduce.

Ribozymes provide another way to lower gene expression levels by damaging a target gene or transcript. However, designing ribozymes to knock out the expression of specific genes may involve significant research and development.

Antisense technology has been shown to be an effective means of down-regulating expression of specific genes. It has been widely used to interfere with eukaryotic gene expression through injection of synthetic oligonucleotides complementary to mRNA (Agrawal et al., Proc. Natl. Acad. Sci. USA, 1997, 94:2620-2625; and Zamecnik et al., Proc. Natl. Acad. Sci. USA, 1978, 75:280-284) and double-stranded RNA (Fire et al., Nature, 1998, 391:806-810), and the synthesis of antisense RNA from DNA cloned in an antisense orientation (Beauregard et al., EMBO J., 1995, 14:409-421; Kernodle et al., Infect. Immun., 1997, 65:179-184; and Ottavio et al, Virology, 1992, 189:812-816). Much research has been done using antisense RNA, and clinical trials for treatment of serious human diseases, such as CMV retinitis, cancer, and HIV infection, are in progress (Cagnon et al., J. AIDS Hum. Retrovirol., 1995, 9:349-358; Ratajczak et al., Proc. Natl. Acad. Sci. USA, 1992, 89:11823-11827; and J. L. Whitton, Adv. Virus Res., 1994, 44:267-303). However, antisense technology has rarely been used to inhibit gene expression in bacteria due to the availability of more powerful techniques, such as directed mutagenesis and homologous recombination, even though there is evidence that antisense regulation occurs naturally in bacteria during plasmid, phage, and chromosomal replication (Van der Krol et al., Biotechniques, 1988, 6:958-976; Wagner et al., Ann. Rev. Microbiol., 1994, 48:717-742). Recent reports have indicated that antisense RNA can inhibit expression of known genes in bacteria by using synthetic antisense RNA from DNA cloned in the reverse orientation (Kemodle et al., Infect. Immun., 1997, 65:179-184) and by peptide nucleic acid (PNA) targeted to mRNA (Good et al., Nature Biotechnology, 1998, 16:355-358).

The identification of virulent and essential genes for bacterial survival in vivo is a powerful approach for studying molecular pathogenesis and determining molecular targets for antibiotic discovery. The combination of an antisense strategy with a regulatory expression system may offer a useful method of studying the molecular pathogenesis of bacterial pathogens. An aspect of the invention herein was to define novel virulence factors by inducing antisense RNA to decrease the expression of known genes during different stages of infection and to target essential genes in vitro and in vivo. Indeed, the Tn10-encoded tet repressor has been successfully used to regulate expression of specific genes not only in Bacillus subtilis (Geissendorfer et al., Appl. Microbiol. Biotechnol., 1990, 33:657-663), but also in mammalian cells (Grossen et al., Proc. Natl. Acad. Sci. USA, 1992, 89:5547-5551; Grossen et al., Science, 1995, 268:1766-1769), transgenic mice (Kistner et al., Proc. Natl. Acad. Sci. USA, 1996, 93:10933-10938) and tobacco (Gatz et al., Mol. Gen. Genet., 1991, 227:229-237). One inducible promoter system that has been shown to be effective in Gram-positive organisms is the xyl/tet chimeric promoter (Geissendorfer et al., Appl. Microbiol. Biotechnol., 1990, 33:657-663). This promoter system employs elements of both the xylose and tetracycline systems and has been shown to be strongly inducible in B. subtilis using sub-inhibitory concentrations of tetracycline. As an example of a preferred embodiment of the invention, a tet regulatory system in Staphylococcus aureus was constructed and cloned an antisense hla fragment downstream of the inducible xyl/tet promoter-operator fusion to investigate whether this tet regulatory system can function in S. aureus in vitro and in vivo and whether inducible antisense hla downregulates expression of the chromosomal hla gene.

The present invention provides methods for creating libraries of conditionally expressed and conditional lethal mutant bacteria and other cells using an antisense strategy (for a description of antisense polynucleotides used in bacteria, see Kerndole, et al., Infection and Immunity 65(1): 179 (1997)). Methods are also provided which allow for the determination of whether a particular gene is essential to the growth or life of the organism being tested. Such genes are particularly useful as targets for screening for antimicrobial compounds.

### SUMMARY OF THE INVENTION

The invention provides a method for determining gene essentiality comprising the steps of: transforming a host cell or group of host cells with a vector comprising an inducible gene control region expressibly linked to a library of random DNA fragments; inducing the inducible gene control region with an inducer; and detecting an alteration in the metabolism of the host cell or group of host cells due to antisense expression.

A method is also provided for determining gene essentiality comprising the steps of: transforming a host cell or group of host cells with a vector comprising an inducible gene control region expressibly linked to an antisense polynucleotide sequence; inducing the inducible gene control region with an inducer; and detecting an alteration in the metabolism of the host cell or group of host cells.

Further provided by the invention is a method for determining gene essentiality comprising the steps of: transforming a host cell or group of host cells with a vector comprising an inducible gene control region expressibly linked to random antisense polynucleotide sequences; inducing the inducible gene control region with an inducer; and detecting killing or slowed growth of the host cell or group of host cells.

A method is still further provided for determining gene essentiality comprising the steps of: transforming a host cell or group of host cells with a vector comprising an inducible gene control region expressibly linked to a library of random DNA fragments; inducing the inducible gene control region with an inducer; detecting an alteration in the metabolism of the host cell or group of host cells; and isolating the full length gene that comprises the coding sequence of the selected polynucleotide sequence or comprises the coding sequence of the complementary sequence of the selected polynucleotide sequence.

The methods of the invention may comprise a host cell as defined elsewhere herein.

The methods may comprise an inducible promoter or an operator and inducible repressor.

The methods may also comprise a selected polynucleotide sequence that is an antisense sequence.

Selected polynucleotide sequences in the methods may be selected from an organism.

Preferred inducers of the invention comprise a chemical compound or electromagnetic radiation. Such chemical compound inducers include, for example, IPTG, doxycycline, erythromycin, and tetracycline. Such electromagnetic radiation includes, for example, X-rays, gamma rays, beta rays, UV light, and visible light, red visible light and green visible light.

Methods are also provided wherein the alteration in the metabolism is slowed cell growth, cell death, or cell stasis.

Various changes and modifications within the spirit and scope of the disclosed invention will become readily apparent to those skilled in the art from reading the following descriptions and from reading the other parts of the present disclosure.

### BRIEF DESCRIPTION OF THE DRAWINGS

FIG. 1 shows a schematic of the tetracycline inducible shuttle vector pYJ90. Abbreviations: tetR, tetracycline resistant repressor-encoding gene; P_{R}, the improved tetR promoter; Pxyl/tetO, the xyl-tet promoter-operator fusion (Geissendorfer et al., Appl. Microbiol. Biotechnol., 1990, 33:657-663); cat, chloramphenicol acetyltransferase-encoding gene; Ap, the ampicillin resistance determinant; Erm, the erythromycin resistance determinant. pUC19 ori and pE194 ori, origins of replication from pUC19 and pE194, respectively, allowing plasmid replication in Gram-negative and Gram-positive host bacteria.

FIG. 2 shows dependence of CAT activity on tetracycline concentration. S. aureus YJ335 was incubated in TSB with 5 ng/ml of Erm to early log phase and different doses of tetracycline were added to aliquotted cultures. Two milliliters of each culture were transferred into a new tube and the cells were harvested by centrifugation three hours after the addition of tetracycline. Crude protein preparations were used to anaylze CAT activity. Specific CAT activity is defined as units of CAT activity per milligram of total protein.

FIG. 3 shows kinetics of tetracycline induction in S. aureus. Strain YJ335 was incubated to early log phase in TBS and 250 ng/ml of tetracycline was added to the culture. Two milliliters of culture were collected 0, 1, 2, 3 and 4 hours after addition of tetracycline. Crude protein extracts of bacteria were prepared and specific CAT activity was determined.

FIG. 4 shows construction of the tetracycline-inducible shuttle vector containing hla in the antisense and sense orientations. A 621-bp fragment of hla containing the promoter region was inserted into the EcoRV site of plasmid pYJ335. Two recombinants, pYJ318-7 and pYJ318-16, represent hla cloned in the antisense and sense hla orientations, respectively.

FIG. 5 shows Northern blot analysis of sense hla and antisense hla transcription. Digoxigenin-labeled single-stranded DNA oligonucleotide probes hybridized specifically with either induced sense hla RNA (YJ318-16+Tc) or induced antisense hla RNA (YJ318-7+Tc).

FIG. 6 shows Western blot analysis of α-hemolysin expressed in strain WCUH29 and its isogenic strains with or without tetracycline induction. The molecular weight markers are biotinylated low molecular weight SDS-PAGE standards (Bio-Rad Lab., Hercules, Calif.).

FIG. 7 shows RT-PCR analysis of transcription of cat (A) and hla (B) following in vivo induction with tetracycline. Plasmid DNA was used as a positive control (pYJ335 and pYJ318-16). Negative controls were samples prepared without RT or template DNA.

### Glossary

The following definitions are provided to facilitate understanding of certain terms used frequently herein.

“Antisense polynucleotide” means a polynucleotide sequence that is capable of hybridizing to or is complementary to, in whole or in part, another polynucleotide sequence.

“Expressibly linked” means a first polynucleotide sequence joined to a second polynucleotide sequence, such as by ligation, so that they act together to express a gene product, such as a DNA, an RNA or a protein.

“Host cell” is a cell which has been transformed or transfected, or is capable of transformation or transfection by an exogenous polynucleotide sequence and includes a cell or cells of a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans. Herein these cells are also referred to as “organisms.”

“Inducer” means a composition of matter or electromagnetic radiation to which an inducible gene control region responds by altering the expression of an expressibly linked polynucleotide. Examples of inducers include those well known in the art, such as UV radiation and IPTG, as well as those disclosed herein.

“Inducible gene control region” means a polynucleotide sequence that responds to a composition of matter or electromagnetic radiation and alters the expression of an expressibly linked polynucleotide. Examples of such regions include inducible promoters or derepressible operator/promoters combinations, many of which are well known.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein.

“Polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation, ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter, et al., Meth. Enzymol. 182:626-646 (1990) and Rattan, et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Selected polynucleotide sequence” means an isolated polynucleotide sequence that is complementary, in whole or part, to a target sequence, or is an isolated polynucleotide randomly selected from a pathogen polynucleotide sequence library.

“Target” means a polynucleotide, such as a gene, that is desired to be targeted to determine the effect of altering its expression level on the metabolism or reproduction of the cell comprising such target.

### DETAILED DESCRIPTION OF THE INVENTION

The biochemical basis of many pathogen resistance mechanisms to antimicrobials is now known. These mechanisms alone, or in concert, are responsible for the escalating problem of antimicrobial resistance seen in both hospital and community acquired infections. The principle approach by researchers to overcome these problems has been to seek incremental improvements in existing drugs. While these approaches contribute somewhat to the fight against infection by such resistant pathogens, new approaches are needed.

Knowledge of genes or gene products essential to the growth of an organism can provide a key to the development of treatments of infectious pathogens. Gene knockout studies provide information on the effect of the total absence of a gene product. However, antimicrobial therapies can rarely achieve the complete abolition of activity of a given gene product. Importantly, gene knockouts cannot be created (by simple insertion/deletion mutagenesis, for example) if the gene products are essential to viability in vitro.

The present invention was based, in part, on studies undertaken in order to develop a system whereby essential genes could be studied in vitro and in vivo in a pathogen, particularly a pathogenic bacterium or organism, such as S. aureus. Current methods for evaluating the requirement for a gene product during in vitro growth or establishment or maintainence of infection are labor-intensive and time-consuming, relying on the generation of random or directed null mutations. Such an approach will necessarily omit mutations in essential genes, as these would likely result in non-viable cells.

Expression of antisense fragments to down-regulate gene expression has several advantages over other methods. The use of antisense can be a powerful tool to aid in understanding a gene's function without necessarily completely eliminating its activity. In the case of essential genes, especially, antisense technology allows one to very easily manipulate the expression of a gene in order to observe the consequences of a lethal mutation over time. The ease with which such antisense fragments can be constructed can be directly contrasted with the tedium of constructing null mutations and promoter-down mutations. The antisense technology was combined with an inducible promoter system to selectively induce expression of an antisense hla fragment as an illustrative example of an embodiment of the invention.

It had been shown previously that a 600-bp fragment from the 5′ end of the hla gene was sufficient to cause a 16-fold decrease in α-hemolysin production when expressed under its native promoter in the antisense orientation (Kernodle et al., Infect. Immun., 1997, 65:179-184). A polynucleotide fragment of the invention was made in order to corroborate the earlier work and to validate the inducibility of the promoter system. The xyl/tet hybrid promoter (Geissendorfer et al., Appl. Microbiol. Biotechnol., 1990, 33:657-663) allows tight regulation of downstream genes, with titratable induction. Maximal expression, as monitored by a promoterless cat reporter gene, was obtained when as little as 0.25 nanograms/ml tetracycline (herein “Tc”) was used; higher concentrations of Tc resulted in decreased CAT activity, likely due to the antibiotic activity of Tc.

That the xyl/tet hybrid promoter is functional in S. aureus and demonstrated titratable activity enabled the monitoring of promoter activity over time and set certain parameters. Antisense down-regulation of hla for was examined for several reasons. First, α-hemolysin is a well-characterized secreted protein with ample, accessible tools with which to study it. Second, α-toxin production can be screened on plates and its absence results in a defined effect, namely, attenuation of virulence in vivo. Third, α-hemolysin expression down-regulated by antisense under the control of its own promoter had been previously demonstrated and thus would be a good comparison for certain embodiments of the invention.

The inducible xyl/tet promoter used in this embodiment had been previously shown to be functional in B. subtilis (Geissendorfer et al., Appl. Microbiol. Biotechnol., 1990, 33:657-663). This promoter incorporates elements of both the xylose- and tetracycline-inducible systems, resulting in a tightly-regulated, strong promoter that is induced with low concentrations of tetracycline. In B. subtilis, using 0.4 μg/ml of Tc, 100-fold induction was obtained within three hours. Results exemplified herein using this promoter construct in S. aureus compare with this earlier one: there was observed a 70-fold level of induction with 0.25 ng/ml of Tc in the same time period, as shown by monitoring specific CAT activity over time.

The inducibility of this promoter system, as well as its ability to down-regulate gene expression, was assessed in vitro by cloning a ˜600-bp fragment of the hla gene downstream of the xyl/tet promoter in both the sense and antisense orientations. Both Northern and Western blot analyses confirmed the decreased production of α-toxin when the hla antisense fragment was expressed using 0.25 ng/ml of Tc to induce. The endogenous α-hemolysin product was not observed in these blots because the hla gene is expressed primarily during stationary phase growth; certain preferred samples useful in the invention studies are prepared from early log-phase cells.

This inducible promoter system was then used to selectively induce expression on a gene, α-hemolysin, during in vivo infection. A murine model of hematogenous pyelonephritis was chosen to illustrate certain embodiments of the invention, as it results in a localized kidney infection from which bacteria are readily recovered. Results exemplified herein demonstrate that, using low levels of tetracycline given orally, one can effectively induce expression of α-hemolysin. This result will enable us to examine essential genes in this manner, using different concentrations of inducer to control the levels of antisense expressed, and thus down-regulate expression to different degrees. Only a 2-fold level of virulence attenuation was observed (as determined by enumeration of CFU) between S. aureus strains expressing hla antisense RNA and those not expressing this transcript. This result may be because α-hemolysin activity is not important for virulence in the hematogenous pyelonephritis model of infection.

The inducible system that is an embodiment of the invention allows one to specifically decrease or abolish expression of a particular gene at will. Therefore, the effects of the absence of the gene product can be studied after synchronization of the cells by the addition of inducer. In addition, the titratability of this promoter system makes it possible to observe the effects of different levels of down-regulation of an essential gene without completely inactivating it. This type of analysis can aid in the development of antimicrobial agents by decreasing levels of a target gene product and perhaps rendering cells more susceptible. In a preferred embodiment of the invention this technology may be applied to a more random approach, i.e. the development of a conditional-lethal screen for essential genes, under both in vitro and in vivo conditions.

The present invention provides a convenient, rapid and cost-effective method for the creation of mutants as a result of conditional gene down-regulation. Certain of these mutants are conditional lethal mutants. The present invention also provides a method for determining pathogen sensitivity to varying levels of reduction of a gene product and is applicable to genes essential in vitro since reduction in levels of the gene product only occurs under conditions of induction. The decrease in gene expression for a selected target can be monitored and correlated with the progression of the infection and/or viable counts recovered from infected tissue. Using this method, genes from a selected pathogen which are most sensitive to inhibition in vivo can be identified and selected as targets for the development of new intervention therapies.

A conditional lethal method is also provided for identifying essential genes using antisense technology. Preferred methods are also provided for using an inducible promoter(s) to selectively express antisense library clones. Colonies that fail to grow under these conditions are likely to be carrying plasmids comprising insert DNA which, when expressed, produce an antisense RNA fragment that inhibits translation of an essential gene transcript. This strategy facilitates identification of such essential genes as they can be readily identified by sequencing the plasmid inserts. In addition, this strategy can be used to screen for both in vitro and in vivo essential genes.

Preferred methods of the invention comprise inducible promoter(s), examples of which are set forth elsewhere herein.

Preferred methods of the invention also comprise shuttle plasmids which can replicate in both Escherichia coli and Staphylococcus aureus. A library of DNA fragments may be cloned into the shuttle plasmids of the invention. An example of a library of DNA fragments useful in the methods and/or in the plasmids of the invention preferably comprise 600 to 1000 base pair fragments.

These methods have significant advantages over those methods presently available and known in the art, including for example, the methods of the invention (i) allow for evaluation of partial gene repression, (ii) facilitate identification of essential genes as the cloned antisense fragment can be used as a probe for the full-length gene sequence, (iii) pools of mutants can be examined en masse in vivo, (iv) after infection, clones which are not recovered (identified by subtractive hybridization) are likely to contain antisense fragments corresponding to in vivo essential genes, (v) antisense effect can be measured directly, by determining levels of antisense expression and correlating with degree of in vivo attenuation, and (vi) can possibly also identify genes for which a partial down regulation is lethal by varying levels of inducer.

By “pathogen” it is meant any organism which is capable of infecting an animal or plant and replicating its nucleic acid sequences in the cells or tissue of the animal or plant. Such a pathogen is generally associated with a disease condition in the infected animal or plant. Such pathogens may include, but are not limited to, viruses, which replicate intra- or extra-cellularly, or other organisms such as bacteria, fungi or parasites, which generally infect tissues or the blood. Certain pathogens are known to exist in sequential and distinguishable stages of development, e.g., infection initiation, latent stages, infective stages, and stages which cause symptomatic diseases. In these different states, the pathogen is anticipated to rely upon different genes as essential for survival and pathogenesis. Preferred host cells of the invention are pathogens. The methods of the invention may comprise a host cell as described elsewhere herein.

In one embodiment of the invention, a method is provided whereby total genomic DNA of a pathogen or host cell is isolated. Random fragments of the size 0.6-1 kb are expressibly linked to an inducible expression control sequence. This method will allow for the determination of whether the expression of a sequence, or gene comprising the sequence, is essential for the cell's growth or survival. This essentiality may be tested under various conditions as described herein.

Optimization of the expression of the antisense RNA to inhibit target gene expression may be first carried out in vitro using a standard controllable promoter such as pSpac, TetR, etc., induced by IPTG and tetracycline, respectively. However, other controllable and inducible promoters known in the art, as well as others taught herein, may also be used for this purpose. Promoters specifically induced by the in vitro environment, i.e., the acetyl-CoA-acyltransferase promoters, may also be used for this purpose.

Following optimization, the expression vector may then be introduced into the selected pathogen using standard techniques. Introduction of the vector carrying the antisense polynucleotide sequence construct into the selected pathogen should not affect growth or expression of the target gene in vitro when the antisense expression construct is uninduced. By contrast, one may determine gene essentiality if introduction of the vector carrying the antisense polynucleotide sequence construct into the selected pathogen affects pathogen growth, reproduction or metabolism in vitro when the antisense expression construct is induced by an inducer. This target will be a preferred target for antimicrobial compound screening.

If, after introduction of a host comprising the construct into animal or plant models, the induced expression of a particular antisense RNA results in a reduction in target gene expression, this target is also of interest for cloning followed by antimicrobial compound screening. Levels of gene expression can be monitored by RT-PCR of total mRNA isolated from infected tissue at various times during the infection and correlated with housekeeping gene controls and viable cell counts. Reduction in target mRNA is correlated with infection progression including disease pathology. Luminescence in thin tissue sections allows determination of the numbers of metabolically active pathogens and viable cell counts allow for the prioritization of gene targets for development of therapeutic agents. For example, in those cases where a significant reduction in target RNA, but little effect on viable cell count, is seen, the gene will be considered to be a less attractive target than situations where reduction in viable counts correlates with decreased target mRNA by RT-PCR analysis.

Genes and gene products identified according to the method of the present invention may then be used in the design of therapeutic and diagnostic agents. For example, genes identified in accordance with this method as essential to a selected pathogen in the infection process and proteins encoded thereby may serve as targets for the screening and development of natural or synthetic chemical compounds which have utility as therapeutic drugs for the treatment of infection by this pathogen. As an example, a compound capable of binding to such protein encoded by such gene and inhibiting its biological activity may be useful as a drug component preventing diseases or disorders resulting from the growth of a particular organism. Alternatively, compounds which inhibit expression or reduce expression of an essential gene are also believed to be useful therapeutically.

Conventional assays and techniques may be used for screening and development of such therapeutics. For example, a method for identifying compounds which specifically bind to or inhibit proteins encoded by these gene sequences can include simply the steps of contacting a selected protein or gene product with a test compound to permit binding of the test compound to the protein; and determining the amount of test compound, if any, which is bound to the protein. Such a method may involve the incubation of the test compound and the protein immobilized on a solid support. Still other conventional methods of drug screening can involve employing a suitable computer program to determine compounds having similar or complementary structure to that of the gene product or portions thereof and screening those compounds for competitive binding to the protein. Such compounds may be incorporated into an appropriate therapeutic formulation, alone or in combination with other active ingredients. Methods of formulating such therapeutic compositions, as well as suitable pharmaceutical carriers, and the like are well known to those of skill in the art.

Accordingly, through use of such methods, the present invention is believed to provide targets for screening compounds capable of interacting with these genes, or encoded proteins or fragments thereof, and either enhancing or decreasing the biological activity, as desired. Such compounds are also encompassed by this invention.

Numerous modifications and variations of the present invention are included in the above-identified specification and are expected to be obvious to one of skill in the art. Such modification and alterations to the compositions and processes of the present invention are believed to be encompassed in the scope of the claims appended hereto.

The invention also provides a method for determining gene essentiality comprising the steps of: transforming a host cell or group of host cells with a vector comprising an inducible gene control region expressibly linked to a random polynucleotide sequence; inducing the inducible gene control region with an inducer; and detecting an alteration in the metabolism of the host cell or group of host cells.

A method is also provided for determining gene essentiality comprising the steps of: transforming a host cell or a group of host cells with a vector comprising an inducible gene control region expressibly linked to a random antisense polynucleotide sequence; inducing the inducible gene control region with an inducer; and detecting an alteration in the metabolism of the host cell or group of host cells. The detection step may be carried out by observing alterations in metabolism in the form of killing or slowed growth of the host cell or group of host cells, as the case may be. Sequences whose lowered expression alters the metabolism of the cell are useful candidate targets for antimicrobial compound screening. In view of this, these sequences, and any full length gene coding sequence comprising such sequences, may be isolated using methods well known to the skilled artisan. These methods include, among others, PCR, cloning, sequencing.

The structure of the vectors useful in the methods of the invention, such as those that drive antisense transcription, may take many forms. For example, the vectors may comprise an inducible promoter or an operator and inducible repressor. Using these gene expression control regions one may regulate the level of expression of the antisense transcription unit. Further particularly preferred vector embodiments comprise two inducible gene control regions, each expressibly linked to each terminus of an inserted DNA fragment. Such insertional vectors allow for the insertion of two inducible gene control regions transcribing in opposite directions. These vectors having two promoters and an inserted element are also useful to obtain antisense expression regardless of the insert direction. Whole libraries may be screened using vectors comprising such dual inducible gene expression control regions in the methods of the invention. For example, random fragments of genomic DNA from the selected pathogen are expressibly linked between the two inducible gene expression control regions. The inducible gene expression control regions are each induced by a different inducer. These expression constructs are then randomly ligated into a vector and the vector is introduced into a pool of pathogen host cells. These cells are replica plated on a first medium comprising an inducer for the first inducible gene control region, on a second medium comprising an inducer for the second inducible gene control region, and on a third medium lacking any inducer. Colonies which fail to grow on the first and/or second media but grow on the third medium contain an essential polynucleotide sequence (e.g., a gene or transcript) corresponding to the antisense polynucleotide sequence of the cloned polynucleotide sequence.

Vectors used in the methods of the invention include, for example, any polynucleotide that may be introduced into a pathogen cell, including polynucleotides stably introduced into the host cell's genome. For bacterial host cells preferred vectors of the invention include shuttle plasmids, which can replicate in both gram (−) and gram (+) hosts to facilitate cloning and library construction. Gene expression elements may be engineered into theses vectors in preferred embodiments. For example, particularly preferred embodiments comprise two inducible gene control regions. It is preferred that one such region is expressibly linked to each terminus of the cloned polynucleotide sequence. Such vectors having two promoters, particularly two inducible promoters, are useful to obtain antisense expression regardless of the insert direction. Vectors useful in the invention may comprise transcriptional or translational terminators ligated upstream or downstream of the antisense polynucleotide in order to keep gene expression off until specific induction is desired. Both eukaryotic and prokaryotic terminators are known in the art.

Vectors of the invention may contain random sequences or may be used to introduce a sequence that has been partially characterized, such as by RT-PCR data. This targeted approach may be used to complement known methods for carrying out gene essentiality analyses, such as STM and RT-PCR. This approach would allow the skilled artisan to examine a limited number of clones in vivo, thereby reducing the complexity of the library being screened by orders of magnitude.

Inducers of the inventions may be any compound or EMR that can induce gene expression driven by a polynucleotide sequence, preferably driven by a promoter. Preferred inducers of the invention comprise a chemical compound or electromagnetic radiation.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., BASIC METHODS IN MOLECULAR BIOLOGY, (1986) and Sambrook et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include pathogens, such as bacterial cells, preferably streptococci, staphylococci, enterococci E. coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used to produce the polypeptides of the invention. Such vectors include, among others, chromosomal, episomal and virus-derived vectors, e.g., vectors derived from bacterial plasmids, from genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

Other useful vectors include, for example, pCU1 (Gram-negative to Gram-positive shuttle vector), pWM401 (Gram-negative to Gram-positive shuttle vector), pHV33 (Gram-negative to Gram-positive shuttle vector), pHV1431 (Gram-negative to Gram-positive shuttle vector), pNZ12 (Gram-negative to Gram-positive shuttle vector). Preferred vectors and related references are listed in Table 1.

TABLE 1

| Plasmid | Description | Related Reference |
|---|---|---|
| pMH109 | contains multiple cloning sites | Hudson et al., Gene, |
| | upstream of the cat gene | 1986, 48: 93-100 |
| pWH353 | contains the tet regulatory | Geissendorfer et al., |
| | elements including the tetR gene | Appl. Microbiol. |
| | and its promoter with a poly-A | Biotechnol., 1990, |
| | block, and the xyl/tet promoter- | 33: 657-663 |
| | operator fusion | |
| pYJ82 | contains the cat gene cloned into | Yanisch-Perron et al., |
| | the EcoRI and BamHI sites of | Gene, 1985, |
| | pUC19 | 33: 103-119 |
| pYJ90 | contais the origin of replication | Horinouchi et al., |
| | from plasmids pE194 and pUC19, | J. Bacteriol., 1982, |
| | which allows replication in Gram- | 150: 804-814 |
| | positive and Gram-negative | |
| | bacteria; contains Erm and Ap | |
| | resistance markers and a multiple | |
| | cloning site | |
| pYJ101 | contains the tet regulatory element | Described herein |
| | inserted into the Clal and HindIII | |
| | sites of pBluescript II KS | |
| | (Stratagene, La Jolla, CA) | |
| pYJ103 | contains the cat gene cloned into | Described herein |
| | EcoRI and PstI sites of pYJ101 | |
| pYJ335 | contains the tet regulatory element | Described herein |
| | and the cat gene cloned into the | |
| | SalI site of pYJ90 | |
| pYJ318-7 | contains a 621-bp hla fragment in | Described herein |
| | the antisense orientation cloned | |
| | into the SmaI site of pYJ335 | |
| pYJ318-16 | contains the hla fragment in the | Described herein |
| | sense orientation cloned into the | |
| | SmaI site of pYJ335 | |

The methods of the invention may be used with any Gram+plasmid made into a shuttle vector by ligation with pBluescript. The skilled artisan will be readily able to make such vectors based on the teachings herein and in the art.

Inducible promoters useful in the methods of the invention may be any inducible promoter, for example, a doxycycline inducible promoter (see Kistner et al., PNAS USA 93: 10933 (1996)), erythromycin resistance promoter (see Ross et al., Gene 183: 143 (1996)), a macrolide resistance promoter (see Shuwsei et al., Antimicrobial Agents and Chemotherapy 41(3): 530 (1997), or a tetracycline resistance promoter (see Geissendorfer et al., Appl. Microbiol. Biotchnol. 33:657-663 (1990); Gossen et al, Science 268: 1766 (1995)); an IPTG inducible promoter, such as pSpac; or an in vivo induced promoter, such as acetyl-CoA-acyltransferase promoter, identified by in vitro expression work by RT-PCR.

Termination sequences useful in the invention include, for example, rho-dependent termination signal, S. aureus and S. pneumoniae termination signals, rho-independent termination.

The invention further relates to packs and kits comprising one or more containers, preferably rigid, filled with one or more of the ingredients of the aforementioned compositions, such as the vectors, of the invention.

Each reference disclosed herein is incorporated by reference herein in its entirety. Any patent application to which this application claims priority is also incorporated by reference herein in its entirety.

### EXAMPLES

### Example 1

Expression of antisense RNA can be a useful tool for studying molecular pathogenesis. In order to induce antisense RNA with the goal of downregulating virulence determinants and essential genes, a tet regulatory expression system was constructed in a shuttle vector. In S. aureus, this regulatory system showed a 70-fold level of induction in vitro and very strong dose dependence; it also functioned in vivo in a murine model of hematogenous pyelonephritis in combination with induction by oral administration of tetracycline. To determine whether induced antisense RNA could interfere with chromosomal gene expression, a 621-bp fragment of the alpha-toxin gene (hla) was cloned downstream of this inducible promoter in antisense orientation, and was transduced into a clinical isolate of S. aureus. Antisense hla RNA inhibited expression of hla in S. aureus and showed a 14-fold decrease compared to the control. These results suggest that the tet regulatory system in S. aureus functions in vitro as well as in vivo and induced antisense RNA can downregulate chromosomal gene expression.

### Example 2

### Bacterial Strains and Plasmids

Certain plasmids useful in the method of the invention are listed in Table 1. S. aureus RN4220 was derived from chemical mutagenesis of S. aureus 8325-4 and is able to accept heterologous DNA (R. P. Novick, Molecular Biology of the Staphylococci. VCH Publishers, New York, N.Y., 1990, 1-40). S. aureus WCUH29 is a virulent alpha-toxin producing clinical isolate. S. aureus strains were cultured in tryptic soy broth (TSB; BBL) or TSB-agar medium. To maintain selection of plasmid pYJ90, S. aureus was grown in culture medium containing erythromycin (Erm 5 μg/ml). Escherichia coli strains were grown in Luria-Bertani broth (LB) containing chloramphenicol (Cm 20 μg/ml), Erm (300 μg/ml), or ampicillin (Ap 100 μg/ml) as appropriate.

### Example 3

### Construction of E. coli-S. aureus Shuttle Vector pYJ90

In order to construct a suitable shuttle vector, plasmids pUC19 (Yanisch-Perron et al., Gene, 1985, 33:103-119) and pE194 (Horinouchi et al., J. Bacteriol., 1982, 150:804-814) were digested with NdeI, purified, ligated, and transformed into E. coli DH5-α by electroporation. Transformants were selected on LB-agar containing Ap (100 μg/ml) and Erm (300 μg/ml). One recombinant, pYJ90, was confirmed by restriction enzyme digestion and electroporated into S. aureus RN4220, as previously described (Kraemer et al., Cur. Microbiol., 1990, 21:373-376 and R. P. Novick, Molecular Biology of the Staphylococci. VCH Publishers, New York, N.Y., 1990, 1-40). Transformants were selected on TSA containing Erm (5 μg/mI). The stability of plasmid pYJ90 in S. aureus was determined by passaging a culture six times in medium lacking antibiotics and analyzing plasmid DNA in the bacterial culture.

### Example 4

### Construction of a tet Regulatory System in Plasmid pYJ90

The ClaI-HindIII fragment containing the tetR gene (which encodes the tet repressor), its promoter (P_{R}), and the strong xyl/tet promoter-operator fusion (P_{xyl/tetO}) was excised from plasmid pWH353, and cloned into plasmid pBluescript II KS (Stratagene, La Jolla, Calif.). The resulting plasmid, pYJ101, was digested with EcoRI and PstI and ligated to the EcoRI-PstI fragment of pYJ82 containing a promoterless cat gene followed by a transcriptional terminator. This new construct was named pYJ103 and the fragment containing the tetR/P_{R}/P_{xyl/tetO}-cat region was cloned into pYJ90 via the SalI site. The resulting plasmid, PYJ335, was confirmed by restriction enzyme digestion and DNA sequencing, and then electroporated into S. aureus RN4220. One of transformants, YJSB335, was confirmed and used to make phage lysates using S. aureus phage φ11.

### Example 5

### Construction of Plasmid pYJ335 Containing Antisense hla and Sense hla

A 621 bp hla fragment was generated by PCR amplification using primers hlaFor64 (5′ GGGGGGCCCGGGTATGTCTTTTCCTTGTTTCA 3′)[SEQ ID NO:1] and hlaRev684 (5′GGGGGGCCCGGGATCAGGTAGTTGCAACTG 3′) [SEQ ID NO:2] corresponding to nucleotides 64-83 and 684-701, respectively. Boldface nucleotides correspond to the SmaI restriction enzyme recognition site and underlined nucleotides correspond to the hla coding sequence (Gray et al., Infect. Immun., 1984, 46:615-618). The amplified hla fragment contains the hla promoter region. The PCR product was digested with SmaI and ligated downstream of the xyl/tetO promoter-operator fusion of pYJ335. The resulting plasmids, pYJ318-7 and pYJ318-16, which contain hla in the antisense and sense orientations, respectively, were separately electroporated into S. aureus. Transformants YJSB318-7 and YJSB318-16 containing antisense hla and sense hla, respectively, were confirmed and used to make φ11 phage lysates.

### Example 6

### S. aureus Transductions

As the clinical isolate, WCUH29, cannot be electroporated, plasmids pYJ318-6 and pJY318-16 were introduced into this strain by phage transduction. Phage φ11 was used to make phage lysates by infecting S. aureus YJSB335, YJSB318-7, and YJSB318-16 grown in top agar (TSB containing 0.7% agar and 5 mMCaCl₂). The phage lysates were sterilized by passing each through a 0.45 μm pore size filter and titered on S. aureus RN4220. Transductions were performed by incubating 5×10⁹ CFU of WCUH29 cells with 100 μl of phage lysate (10⁹-10¹⁰ pfu) and 5 mM CaCl₂ at 37° C. for 30 minutes. One milliliter of ice-cold 20 mM sodium citrate was added to the above mixture to block phage adsorption. The bacterial cells were spun down and resuspended in 500 μl of 20 mM sodium citrate. Transductants were selected on TSB-agar containing 500 μ/ml of sodium citrate and 5 μg/ml of Erm, and transductants YJ335, YJ318-7, and YJ318-16 containing plasmids pYJ335, pYJ318-7, and pYJ318-16, respectively, were confirmed by restriction enzyme digestion.

### Example 7

### PCR, RT-PCR, and DNA Sequencing Techniques

The 621-bp hla fragment was generated by PCR using hla-specific primers. The antisense hla and sense hla orientations in plasmids pYJ318-7 and pYJ318-16, respectively, were confirmed by PCR using the plasmid-specific primer tetRFor 1399 (5′ CAATACATTGTAGGCTGC 3′) [SEQ ID NO:3] corresponding to nucleotides 1399-1416 and hla-specific primers hlaRev684 (reverse) and hlaFor64 (forward). The reaction conditions for all PCR's were 0.2 mM dNTPs, 2.5 mM MgCl₂, 50 pmol of each primer, and 2.5 U of Taq polymerase in buffer supplied by the manufacturer (Gibco-BRL, Cockeysville, Md.). For the antisense hla and sense hla orientations, the primers were tetRFor 1399 and hlaFor64, and tetRFor 1394 and hlaRev684, respectively, using the same annealing temperature of 48° C. For RT-PCR analysis, bacterial RNA was isolated from infected tissue samples using FastRNA reagents (BIO101, Vista, Calif.) and treated with RNase-free DNaseI (GeneHunter Corp., Nashville, Tenn.) to remove DNA. Single-stranded cDNA was synthesized by incubating Dnase-treated RNA with reverse transcriptase in reaction buffer supplied by the manufacturer (Gibco-BRL, Gaithersburg, Md.). After RnaseH treatment, cDNA was used as the template for PCR using the tetR-cat-specific primers, tetRFor 1399 and catRev768 (5′ GGCAGGTTAGTGACATTAG 3′) [SEQ ID NO:4], and the hla gene-specific primers, hlaFor64 and hlaRev684. DNA sequencing was performed to further confirm the tet regulatory elements in pYJ335, and the antisense hla and sense hla orientations in pYJ318-7 and pYJ318-16, respectively.

### Example 8

### Specific CAT Activity Assays

CAT activity was determined spectrophotometrically as described by Shaw (W. V. Shaw, Methods Enzymol., 1975, 43:737-755) using kinetic SoFTmax PRO II software (Molecular Devices Corp. Sunnyvale, Calif.) to monitor activity. Briefly, S. aureus YJ335 was grown with shaking in TSB-Erm at 37° C. to A₆₀₀=0.25. The culture was divided, and different doses (0, 2.5, 25, 250, 500, 1000 ng/ml) of Tc were added to the cultures. Two milliliters were removed from each culture 3 hours after the addition of Tc for the dose-dependent assay, or after 0, 1, 2, 3, and 4 hours following the addition of Tc for the time-course assay. The bacterial cells were harvested by centrifugation and washed once with 25 mM Tris pH 7.8, 10 mM EDTA (TE) buffer. Crude protein extracts were prepared by centrifugation after the bacterial cells had been suspended in 200 μl of TE buffer containing 0.2 mg/ml of lysostaphin (Sigma, St. Louis, Mo.) and incubated at 37° C. for 10 minutes. The total protein concentration was determined by using the Bio-Rad protein microassay (Bio-Rad Lab., Hercules, Calif.). Specific CAT activity was calculated as the number of units of CAT activity per mg total protein. Experiments were performed in triplicate at lease twice and similar results were obtained.

### Example 9

### Northern Blot Analysis

S. aureus YJ318-7 and YJ318-16 were grown in TSB-Erm to an A₆₀₀ of 0.25 with and without Tc (250 ng/ml) and total RNA was extracted using a Qiagen RNeasy mini protocol kit (Qiagen, Inc, Chartsworth, Calif.). The RNA was separated by electrophoresis on a 1.2% agarose, 1.8% formaldehyde gel and blotted onto a nylon membrane (Boehringer Mannheim Biochemicals, Indianapolis, Ind.). RNA was cross-linked to the membrane by UV irradiation using an UV Stratalinker (Stratagene, La Jolla, Calif.). Blots were prehybridized and then hybridized with DIG-labeled single-stranded DNA oligonucleotides in high SDS buffer (Boehringer Mannheim Biochemicals, Indianapolis, Ind.) at 50° C. for 6 hours. Single-stranded DNA oligonucleotides specific for either sense hla RNA (5′GGCCAGGCTAAACCACTTTTGTTAGCACCTTCTTCGCTATAAACTCTATA 3′) [SEQ ID NO:5] or antisense hla RNA (5′TATAGAGTTTATAGCGAAGAAGGTGCTA ACAAAAGTGGTTTAGCCTGGCC 3′) [SEQ ID NO:6] were labeled by 3′ tailing digoxigenin-dUTP (Boehringer Mannheim Biochemicals, Indiannapolis, Ind.) and 100 pmol of each was used to probe the membranes. The DIG-DNA-RNA hybridization was detected by enzyme immunoassay with luminescence (Boehringer Mannheim Biochemicals, Indiannapolis, Ind.) and exposed to X-ray film.

### Example 10

### Western Blot Analysis

For preparation of extracellular protein, Tc was added to 10-ml cultures of S. aureus WCUH29, YJ318-7 and YJ318-16 to a final concentration of 250 ng/ml and incubated with shaking at 37° C. for 8 hours. Supernatants were collected after centrifugation and transferred into tubes containing an equal volume of ethanol and incubated overnight at 4° C. Extracellular proteins were precipitated by centrifugation at 15,000×g at 4° C. for 30 minutes. SDS-PAGE and Western blotting methods used were performed as previously described (U. K. Laemmli, Nature, 1970, 227:680-685). Equal amounts of protein were loaded into each lane of a 12.5% SDS-PAGE gel. Standard α-hemolysin and anti-rabbit antibody alkaline phosphatase conjugate were from Sigma (St. Louis, Mo.). Western blots were scanned using Eagle Eye-II software (Stratagene, La Jolla, Calif.) to quantitate protein bands.

### Example 11

### Murine Hematogenous Pyelonephritis Infection Model

CD-1 female mice (25 g) obtained from Charles River Laboratories were used for in vivo assays. S. aureus YJ335 and YJ318-16 were harvested from 1 ml of stationary phase culture, washed once with 1 ml of PBS, and diluted to an A₆₀₀ of 0.2. These bacterial suspensions were diluted and plated onto TSB-agar plates for determination of viable CFU. Three mice per group were infected with about 10⁷ CFU of bacteria via an intravenous injection of 0.2 ml of bacterial suspension into the tail vein using a tuberculin syringe. Different doses of Tc were given orally in 0.2 ml doses to infected mice on days 1, 2, and 3 after infection. The mice were sacrificed by carbon dioxide overdose 2 hours after the last dose of Tc induction. Kidneys were aseptically removed and each pair was cleaved in half; one half was snap-frozen in cryovials in liquid nitrogen, and the other half was homogenized in 1 ml of PBS for enumeration of viable bacteria. The frozen samples were subsequently used for RT-PCR analysis.

### Example 12

### Construction of the tet Regulatory System in S. aureus

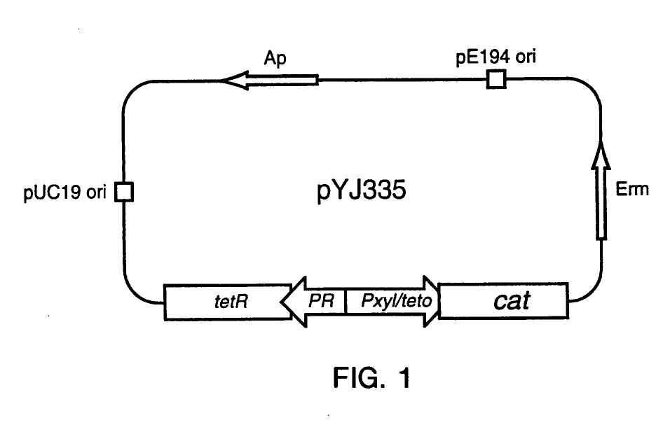

In order to inhibit expression of an endogenous chromosomal gene using induced antisense RNA, the Tn10-encoded tet regulatory element was used as an inducible expression system in S. aureus. This system consists of the tetR gene, the tetR promoter, and the strong xyl/tet promoter-operator fusion to direct expression of the cat gene as a reporter to monitor the level of induction (FIG. 1). The tet regulatory elements and the cat gene were cloned into the E. coli-S. aureus shuttle vector pYJ90 as described elsewhere herein. The resulting plasmid, pYJ335, was found to be stably maintained in S. aureus following multiple passages in the absence of selection (data not shown).

### Example 13

### Establishment of Tc Dose and Timing of Induction

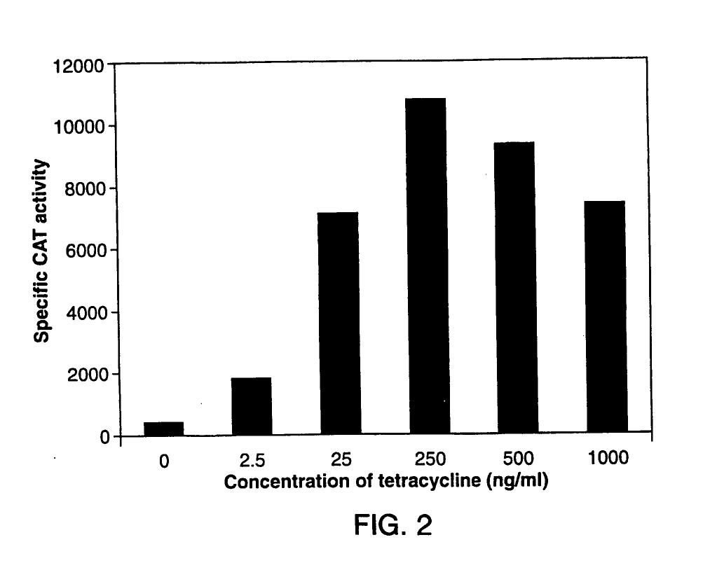

To confirm the function of the tet regulatory expression system in S. aureus, specific CAT activity in strain YJ335 was determined in vitro following induction with tetracycline. The effect of Tc on the expression of cat in strain YJ335 was measured 3 hours after incubation with different doses of Tc in log phase cultures. The results of this experiment are shown in FIG. 2. In the absence of tetracycline, strain YJ335 showed a basal level of cat activity and could not grow on TSB-agar plates containing Cm (1 μg/ml). However, cat expression was induced efficiently when Tc was added to the culture medium. Maximal specific cat activity was 10753 U 3 hours after the addition of 250 ng/ml Tc, whereas a lower dose (25 ng/ml) or a higher dose (500 ng/ml) of Tc resulted in 7120 U and 9328 U, respectively. These results indicate that this tet regulator expression system shows a strong dose-dependent induction and little basal level expression without induction in S. aureus.

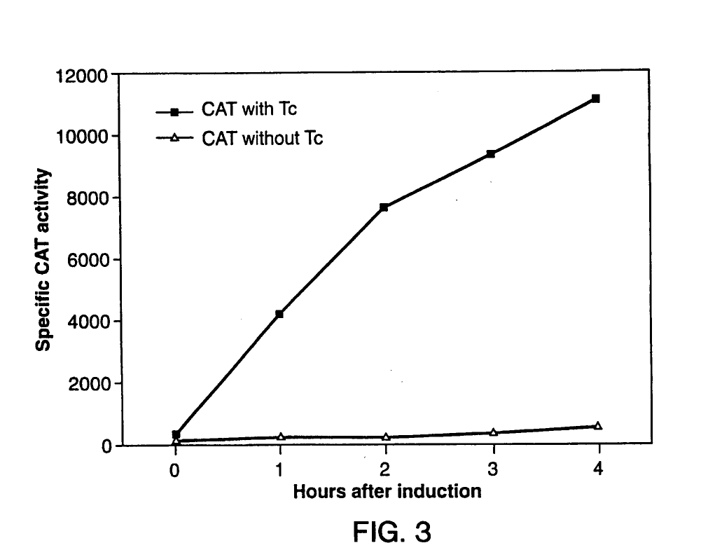

To study the kinetics of induced cat expression in S. aureus strain YJ335, specific CAT activity was measured at 0, 1, 2, 3, and 4 hours after the addition of 250 ng/ml of Tc to a log phase culture. A similar culture grown without tetracycline was used as a control. A 50-fold increase in specific CAT activity was observed after one hour, increased steadily, and by four hours was seen to have increased 70-fold (FIG. 3). In contrast, in the absence of tetracycline, specific cat activity showed a basal level of expression and only increased about 3.7 fold after four hours. These results confirm that this tet regulatory expression system can efficiently regulate the expression of genes downstream of the xyl/tet promoter-operator fusion in S. aureus, resulting in both strong induction and low basal level activity over time.

### Example 14

### Construction of Isogenic Strains Producing Sense and Antisense hla Transcripts in S. aureus WCUH29

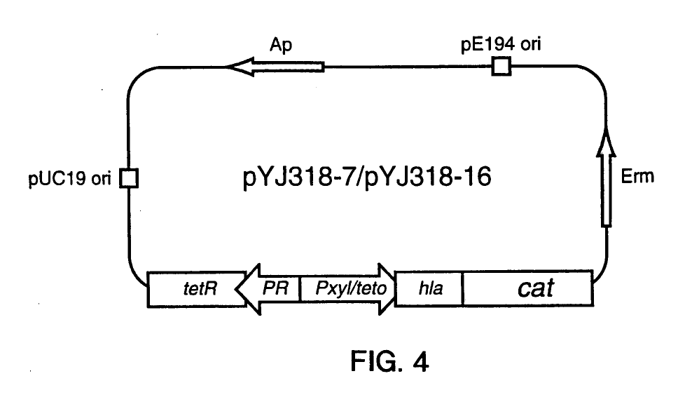

In order to determine whether induced antisense RNA can down-regulate expression of chromosomal genes efficiently in S. aureus, a 621 -bp fragment of the hla gene containing the promoter region was inserted into the SmaI site downstream of the xyl/tet promoter-operator fusion in the shuttle vector pYJ335 (FIG. 4). The orientation of the hla insertion was ascertained by PCR using the plasmid-specific primer tetRFor 1399 and two hla-specific primers hlaFor64 and hlaRev684. Only recombinants containing the hla fragment in the antisense orientation yielded a PCR product of approximately 800 bp using primers tetRFor1394 and hlaFor64, as expected; in contrast, only recombinants containing the hla fragment in the sense orientation produced a PCR product of approximately 800 bp using primers tetRFor1399 and hlaRev684 (data not shown). Two recombinants, pYJ318-7 and pYJ318-16, which contain the 621-bp hla fragment in the antisense and sense orientations, respectively, were electroporated into RN4220 separately. Transformants were selected by Erm resistance and plasmids were confirmed using restriction enzyme digestion. The resulting isogenic strains, YJSB318-7 and YJSB318-16, were used to make phage lysates for transduction as described elswhere herein. The resulting transductants were used to characterize hla antisense function in vitro and in vivo.

### Example 15

### Characterization of the Isogenic S. aureus Strains YJ318-7 and YJ318-16

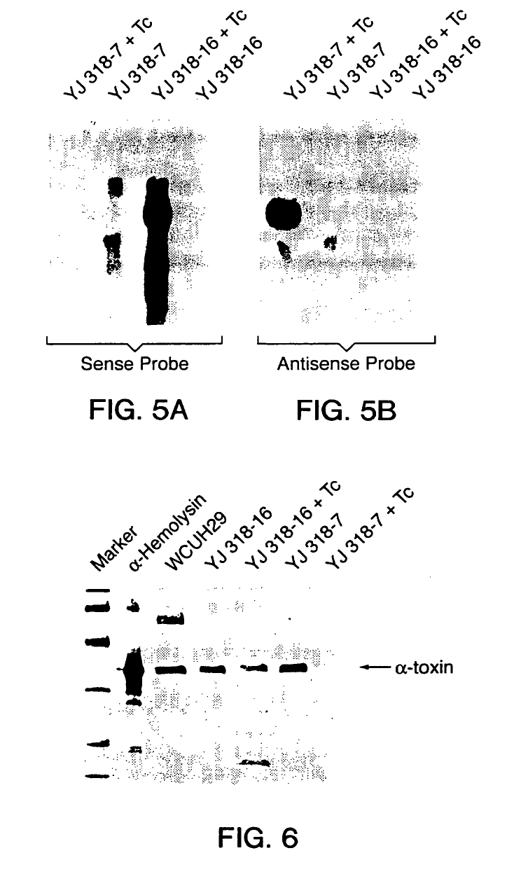

The in vitro expression of sense and antisense hla transcripts was examined by Northern blot analysis after induction with tetracycline. Ten μg of total RNA from YJ3 18-7 and YJ318-16 grown in the presence and absence of 250 ng/ml Tc were electrophoresed on an agarose-formaldehyde gel, blotted onto a nylon membrane, and hybridized to different DIG-labeled single-stranded DNA oligonucleotide probes specific for antisense hla RNA and sense hla RNA (FIG. 5). The blot showed that RNA from strain YJ318-7 after Tc induction contained an antisense hla RNA transcript that hybridized to the antisense DNA probe; in contrast, RNA from strain YJ318-7 without Tc induction did not show the antisense hla RNA transcript. In addition, neither RNA prepared from YJ318-16 with Tc induction nor RNA from YJ318-16 without induction contained the antisense RNA transcript. Only RNA from YJ318-16 with Tc induction contained the sense hla RNA transcript. These results indicate that antisense RNA can be specifically induced with Tc using this tet regulatory expression system in S. aureus and cannot be detected without induction.

To further confirm that induced antisense hla RNA could down-regulate production of the chromosomal hla gene, extracellular proteins in the supernatant of each culture were examined by Western blot analysis using anti-α-hemolysin antiserum. Weak reactivity was observed from Tc-induced YJ318-7 supernatants; in contrast, extracellular proteins in the supernatant of YJ318-7 without Tc induction reacted strongly with the anti-α-hemolysin antibodies (FIG. 6). The difference in α-hemolysin produced by YJ318-7 with Tc induction and without induction was 14-fold when the blot was analyzed using densitometer scanning. In addition, there was no obvious difference in the amounts of α-hemolysin in the supernatants of YJ318-16 with or without Tc induction and wild type WCUH29. These results confirm that antisense RNA induced with Tc using this tet regulatory expression system can efficiently inhibit expression of at least this chromosomal gene in S. aureus.

### Example 16

### In vivo Induction of cat and hla Transcription

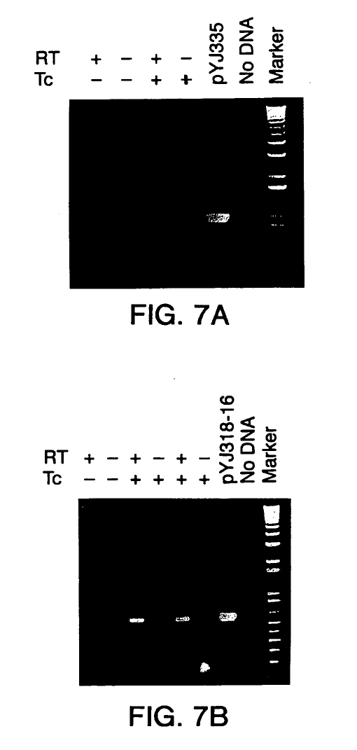

In order to evaluate the function of this tet regulatory expression system in vivo and eventually use Tc to induce antisense RNA during infection, transcription of cat and hla in vivo was measured by RT-PCR after oral Tc induction. The results shown in FIG. 7 indicate that only RNA purified from infected kidneys with Tc induction yielded a specific cat product (FIG. 7A) and a specific hla product (FIG. 7B); no specific RT-PCR product was seen in the infected kidneys without Tc induction. These results suggest that the tet regulatory expression system can be used efficiently ini vivo as well as in vitro to regulate expression of genes downstream of the P_{xyl/tetO} promoter operator fusion in S. aureus.

1. A method for determining gene essentiality comprising the steps of:
transforming a group of host cells with a vector comprising an inducible gene control region expressibly linked to random polynucleotide sequences; inducing said inducible gene control region with an inducer; and detecting an alteration in the metabolism of said group of host cells.

2. A method for determining gene essentiality comprising the steps of:
transforming a group of host cells with a vector comprising an inducible gene control region expressibly linked to random antisense polynucleotide sequence; inducing said inducible gene control region with an inducer; and detecting an alteration in the metabolism of said group of host cells.

3. A method for determining gene essentiality comprising the steps of:
transforming a group of host cells with a library comprising an inducible gene control region expressibly linked to random antisense polynucleotide sequences; inducing said inducible gene control region with an inducer; and detecting killing or slowed growth of said group of host cells.

4. A method for determining gene essentiality comprising the steps of:
transforming a group of host cells with a library comprising an inducible gene control region expressibly linked to random selected polynucleotide sequences;
inducing said inducible gene control region with an inducer; detecting an alteration in the metabolism of said group of host cells; and isolating the full length gene that comprises the coding sequence of a particular polynucleotide sequence or comprises the coding sequence of the complementary sequence of said selected polynucleotide sequence.

5. The method of claim 1 wherein said transforming step said host cell is selected from the group consisting of a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

6. The method of claim 1 wherein said inducible gene control region is an inducible promoter or an operator and inducible repressor.

7. The method of claim 1 wherein said selected polynucleotide sequence is an antisense sequence.

8. The method of claim 1 wherein said selected polynucleotide sequence is from an organism selected from the group consisting of a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

9. The method of claim 1 wherein said vector comprises two inducible gene control regions, one expressibly linked to each terminus of said selected polynucleotide sequence.

10. The method of claim 1 wherein said inducer is a chemical compound or electromagnetic radiation.

11. The method of claim 1 wherein said alteration in the metabolism is slowed cell growth, cell death, or cell stasis.

12. The method of claim 6 wherein said promoter is inducible by an inducer selected from the group consisting of: IPTG, doxycycline, erythromycin, tetracycline, and electromagnetic radiation.

13. The method of claim 7 wherein said antisense sequence comprises the complementary sequence of gene expression control element.

14. The method of claim 13 wherein said gene expression control element is selected from the group consisting of a promoter, an enhancer, and a terminator.

15. The method of claim 9 wherein each of said two inducible control regions is induced by a different inducer.

16. The method of claim 1 wherein said electromagnetic radiation is selected from the group consisting of ultraviolet light, visible light, red visible light and green visible light.
